(19) United States
(12) Patent Application Publication
(10) Pub. No.: US 2026/0248868 A1
(43) Pub. Date: Aug. 27, 2026

(54) COMPOSITION FOR SYNERGISTIC RELIEF OF PHYSICAL FATIGUE AND ITS APPLICATION

(71) Applicant: Xianghu Laboratory (Zhejiang Provincial Laboratory of Agriculture), Hangzhou (CN)

(72) Inventors: Tao Liu, Hangzhou (CN); Guiling Yang, Hangzhou (CN); Jintao Cheng, Hangzhou (CN); Shiyu Li, Hangzhou (CN); Yao Zhao, Hangzhou (CN)

(21) Appl. No.: 19/433,171

(22) Filed: Dec. 26, 2025

(30) Foreign Application Priority Data
Feb. 24, 2025 (CN) 202510200196.7

(51) Int. Cl. A61K 36/258; A61K 36/074; A61K 36/815; A61P 21/00

(52) U.S. Cl. CPC A61K 36/258; A61K 36/074; A61K 36/815; A61P 21/00

(57) ABSTRACT

The invention discloses a composition for synergistically relieving physical fatigue and its application. The composition—comprising at least two components selected from Red ginseng, Lycium ruthenicum, and Ganoderma lucidum, compounded in a specific ratio-synergistically regulates antioxidant capacity, calcium ion metabolism, and energy metabolism. It increases bodily ATP content and glycogen reserves, accelerates the clearance of lactic acid and urea nitrogen, extends exercise duration, and significantly alleviates physical fatigue, with a synergistic effect superior to that of any single component used alone. The composition features simple, all-natural plant-derived components, validated through scientific demonstration and experimental verification. It has a clear mechanism of action and definite interaction among components in fatigue relief, along with the advantages of being green and safe. Additionally, the composition's preparation process is simple and easy to operate, requiring no professional technicians or large-scale equipment. It boasts low production costs and facilitates industrialization.

### CROSS-REFERENCE TO RELATED APPLICATION

This application claims priority to Chinese Invention Patent Application No. ZL202510200196.7, filed with the China National Intellectual Property Administration on Feb. 24, 2025, entitled “A Composition for Synergistic Relief of Physical Fatigue and Its Application”, the entire contents of which are incorporated herein by reference.

### TECHNICAL FIELD

The present invention relates to the field of medicine and food technology, and in particular to a composition for synergistic relief of physical fatigue and its application.

### BACKGROUND ART

Fatigue is a common physiological phenomenon in humans. Exercise-induced fatigue refers to a state where the body fails to maintain function at a specific level or sustain predetermined exercise intensity. Certain food-derived active ingredients can significantly alleviate physical fatigue by regulating energy metabolism and metabolite clearance, via multiple pathways including antioxidation, reduction of metabolite accumulation, anti-inflammation, regulation of energy metabolism, and intestinal microecology modulation.

In recent years, anti-fatigue research has achieved some advances both domestically and internationally, yet numerous unresolved issues remain. For instance, certain known active ingredients offer limited anti-fatigue effects and may pose potential side effects. Many products are formulated based on practical experience, resulting in complex formulas where the selection and proportioning of raw materials lack scientific demonstration and experimental validation. The interaction between components in fatigue-relieving formulas remains unclear, and the underlying mechanisms of compound active ingredient products have not been fully elucidated. Additionally, the formula design and production processes of existing products have inherent limitations, making it difficult to meet market demands. With the continuous enhancement of consumers' health awareness, products developed from natural plants—especially food-medicinal homologous substances—are increasingly recognized for their minimal side effects and health-promoting properties. Therefore, developing scientifically validated, safe, and directly edible physical fatigue-relieving products with synergistic effects, based on food-medicinal homologous substances or natural plant-derived active ingredients, holds significant practical value.

In view of this, the present invention is hereby proposed.

### SUMMARY

The present invention aims to provide a composition for synergistically relieving physical fatigue and its application. The composition comprises at least two components selected from Red ginseng, Lycium ruthenicum, and Ganoderma lucidum, compounded in a specific ratio to effectively alleviate physical fatigue.

### Technical Solution

To achieve the above objective, the technical solution adopted by the present invention is as follows:

The present invention provides a composition comprising at least two components selected from Red ginseng, Lycium ruthenicum, and Ganoderma lucidum.

Red ginseng: A perennial herb of the genus Panax in the Araliaceae family, Red ginseng is a processed product of ginseng. It is prepared by removing the lateral and fine roots of ginseng, followed by steaming, sun-drying, or oven-drying. It contains multiple functional components such as ginsenosides, polyacetylene compounds, and acidic polysaccharides. Among these, ginsenosides are the main active components responsible for anti-fatigue effects, replenishing qi and blood, and improving blood circulation. Red ginseng has long been recognized for its high medicinal value, including replenishing primordial qi, restoring pulse, rescuing from collapse, and tonifying qi to control blood.

Lycium ruthenicum: Also known as black wolfberry, it belongs to the genus Lycium in the Solanaceae family. In traditional Chinese medicine, it is sweet in taste and mild in nature, with a meridian tropism for the Liver and Kidney. It possesses multiple functions, including anti-fatigue, anti-aging, nourishing the liver and kidney, replenishing essence to improve eyesight, invigorating the stomach and nourishing the brain, beautifying the skin, and promoting meridian circulation. Clinically, it is used for conditions such as kidney deficiency with impotence, soreness and weakness of the waist and knees, dizziness, blurred vision, and physical debility.

Ganoderma lucidum: A traditional tonic in Chinese medicine, its main medicinal components include polysaccharides, saponins, flavonoids, and amino acids. Ganoderma lucidum polysaccharide, as a key active component, can enhance immunity, protect the heart and liver, lower blood pressure, promote diuresis, resist aging, and scavenge free radicals. It exhibits multiple functional effects such as anti-tumor, immune regulation, antioxidation, anti-aging, and anti-radiation.

The present invention screens fatigue-relieving ingredients from various food-derived active components by analyzing and comparing their effects on bodily ATP content, glycogen levels, and the clearance of lactic acid and urea nitrogen. Red ginseng exerts anti-fatigue activity by regulating serum urea nitrogen and glycogen content. Lycium ruthenicum polysaccharide, the main active component of Lycium ruthenicum, increases superoxide dismutase (SOD) activity in serum and liver, enhances antioxidant capacity, and relieves fatigue. Ganoderma lucidum polysaccharide increases glycogen reserves in mice, reduces the accumulation of lactic acid and urea nitrogen, and exerts anti-fatigue effects through its strong antioxidant and anti-inflammatory properties, as well as regulating body metabolism. Therefore, Red ginseng, Lycium ruthenicum, and Ganoderma lucidum are selected for binary or ternary compounding. The compound composition exerts a synergistic effect by increasing ATP and glycogen levels and accelerating the clearance of lactic acid and urea nitrogen, thereby significantly enhancing anti-fatigue activity.

### Composition Forms and Sources

The compositions of the present invention include binary combinations (Red ginseng & Lycium ruthenicum, Red ginseng & Ganoderma lucidum, Lycium ruthenicum & Ganoderma lucidum) and a ternary combination (Red ginseng, Lycium ruthenicum, & Ganoderma lucidum).

The components can be used in forms such as powder, extract, soft extract, concentrate, or cut slices. Extracts may be aqueous or alcoholic, and those skilled in the art can adjust the form as needed. In some embodiments, Red ginseng, Lycium ruthenicum, and Ganoderma lucidum are all processed into powder for compounding. The source, preparation, or extraction method of the components is not limited; they can be commercially purchased or prepared by conventional processing methods in the field.

### Optimal Component Ratios

To achieve optimal effects, the present invention experimentally verifies the effects of different component ratios in each combination, confirming that biological activity varies significantly with ratio.

Binary combination (Red ginseng extract powder: Lycium ruthenicum extract powder): 2-8 parts: 1-9 parts by weight. Exemplary ratios include (but are not limited to) 2:1, 2:9, 3:1, 3:9, 4:1, 4:9, 5:1, 5:9, 6:1, 6:9, 7:1, 7:9, 8:1, 8:9, or any ratio within the range of 2-8:1-9.

Binary combination (Red ginseng extract powder: Ganoderma lucidum extract powder): 2-8 parts: 3-10 parts by weight. Exemplary ratios include 2:3, 2:10, 3:3, 3:10, 4:6, 4:10, 5:3, 5:10, 6:3, 6:10, 7:3, 7:10, 8:3, 8:10, or any ratio within the range of 2-8:3-10.

Binary combination (Lycium ruthenicum extract powder: Ganoderma lucidum extract powder): 1-9 parts: 3-10 parts by weight. Exemplary ratios include 1:3, 1:10, 2:3, 2:10, 3:3, 3:2, 4:3, 4:10, 5:3, 5:10, 9:3, 9:10, or any ratio within the range of 1-9:3-10.

Ternary combination (Red ginseng extract powder: Lycium ruthenicum extract powder: Ganoderma lucidum extract powder): 2-8 parts: 1-9 parts: 3-10 parts by weight. Non-limiting ratios include 2:1:3, 2:1:10, 2:9:3, 2:9:10, 8:1:3, 8:1:10, 8:9:3, 8:9:10, or any ratio within the range of 2-8:1-9:3-10.

Preferred Embodiment: The composition comprises 2 parts Red ginseng extract powder, 3 parts Lycium ruthenicum extract powder, and 8 parts Ganoderma lucidum extract powder by weight. The lowest combination index (CI) value indicates that this 2:3:8 mass ratio exhibits the most potent anti-fatigue activity in mice, with optimal modulation of antioxidant capacity, Ca²⁺ metabolism, energy metabolism, and glycogen levels.

### Combination Index (CI) Principle

The combination index is a method for assessing the combined effects of components based on dose-effect curves, particularly suitable for evaluating interactions after compounding two or more components. It judges whether the combination exerts synergistic (CI<1), additive (CI=1), or antagonistic (CI>1) effects by comparing the actual effect with the theoretical effect of combined use. By combining experimental screening of food-derived anti-fatigue candidates with CI analysis, synergistic, performance-enhancing functional food formulas can be rationally designed.

### Experimental Verification

In the present invention, both binary and ternary compositions significantly increased swimming distance and recovery rate, elevated ATP levels, reduced lactic acid accumulation, and upregulated the expression of Nrf2 (oxidative stress pathway), atp2b1 (Ca²⁺ handling), and g6pca (energy metabolism). This demonstrates concerted modulation of antioxidant, calcium, and energy pathways that underpin fatigue relief.

The efficacy of the ternary blend was further validated using the Methods for Functional Inspection and Evaluation of Health Food (2023 Edition, State Administration for Market Regulation of the People's Republic of China). Mammalian tests confirmed prolonged swimming time, lower serum lactic acid and urea nitrogen levels, and higher muscle and hepatic glycogen content, corroborating robust anti-fatigue efficacy.

### Application of the Composition

The present invention provides the use of the above compositions in manufacturing products for alleviating physical fatigue, enhancing ATP energy metabolism, reducing lactic acid levels, or modulating the expression of Nrf2, atp2b1, and g6pca.

With the growing consumer demand for fatigue-management solutions, the compositions offer significant commercial and social value. They can be incorporated into foods, dietary supplements, or pharmaceuticals:

Food products: Processed by conventional techniques, optionally with food-grade excipients (solvents, binders, fillers, sugars, sweeteners, etc.). Suitable formats include tablets, capsules, oral liquids, powdered beverages, pills, granules, decoctions, pastes, syrups, tinctures, drop pills, or sachet teas.

Pharmaceuticals: Formulated as oral preparations, optionally with pharmaceutically acceptable excipients (buffers, emulsifiers, suspending agents, disintegrants, dispersants, binders, vehicles, stabilizers, chelators, diluents, gelling agents, preservatives, wetting agents, lubricants, absorption retardants, or liposomes). Dosage forms may include tablets, oral solutions, hard/soft capsules, granules, powders, or pills.

The incorporation levels of the composition and excipients can be tailored to needs without specific limitations.

### Key Advantages of the Invention

Synergistically regulates antioxidant, calcium, and energy metabolism pathways, increasing ATP and glycogen levels while accelerating the clearance of lactic acid and urea nitrogen. This extends exercise endurance beyond what any single component can achieve.

Features a simple, all-botanical composition with a clear, experimentally verified mechanism and an excellent safety profile.

Enables low-cost manufacturing that does not require specialized personnel or heavy equipment, facilitating rapid industrial scale-up.

### DESCRIPTION OF EMBODIMENTS

To clarify the objectives, technical solutions, and advantages of the present invention, the technical solutions of the embodiments are described in full and detail below. Experiments were conducted under routine laboratory conditions, or in accordance with the manufacturers' instructions if no specific conditions were specified. All reagents and instruments were commercially available conventional products.

The following examples further illustrate the invention. They are intended to provide detailed information only and shall not be construed as limiting the scope of the invention in any way.

Red ginseng, Lycium ruthenicum, and Ganoderma lucidum were purchased from the open market.

Raw Material Preparation (Applicable to All Examples and Comparative Examples)

Qualified batches of each raw material were individually weighed, coarsely crushed, and extracted with water in a multi-functional extractor for 4 hours. The extract was filtered through a 100-mesh sieve, concentrated under reduced pressure to a relative density of 1.06, spray-dried, and the resulting powder was sieved through an 80-mesh sieve to obtain the dry powders used in the examples.

### Compositions

Composition 1: 3 parts Red ginseng extract powder, 4 parts Lycium ruthenicum extract powder

Composition 2: 2 parts Red ginseng extract powder, 9 parts Lycium ruthenicum extract powder

Composition 3: 8 parts Red ginseng extract powder, 1 part Lycium ruthenicum extract powder

Composition 4: 3 parts Red ginseng extract powder, 4 parts Ganoderma lucidum extract powder

Composition 5: 1 part Red ginseng extract powder, 5 parts Ganoderma lucidum extract powder

Composition 6: 8 parts Red ginseng extract powder, 3 parts Ganoderma lucidum extract powder

Composition 7: 3 parts Lycium ruthenicum extract powder, 4 parts Ganoderma lucidum extract powder

Composition 8: 1 part Lycium ruthenicum extract powder, 10 parts Ganoderma lucidum extract powder

Composition 9: 3 parts Lycium ruthenicum extract powder, 1 part Ganoderma lucidum extract powder

Composition 10: 1.5 parts Red ginseng extract powder, 1.5 parts Lycium ruthenicum extract powder, 4 parts Ganoderma lucidum extract powder

Composition 11: 1.5 parts Red ginseng extract powder, 1.5 parts Lycium ruthenicum extract powder, 10 parts Ganoderma lucidum extract powder

Composition 12: 3 parts Red ginseng extract powder, 3 parts Lycium ruthenicum extract powder, 1 part Ganoderma lucidum extract powder

Composition 13: 2 parts Red ginseng extract powder, 3 parts Lycium ruthenicum extract powder, 8 parts Ganoderma lucidum extract powder

### Example 1

Wild-type AB-strain zebrafish larvae at 6 days post-fertilisation (6 dpf) were used. Each composition listed above was dissolved in system water at the same concentration. The larvae were incubated at 27° C. for 24 hours in the presence of the test substance (treatment details are shown in Table 1). Fatigue was then induced by exposure to sodium sulfite for 30 minutes, after which the total swimming distance of each larva within 30 minutes was recorded. The recovery rate, lactic acid content, ATP content, and target gene expression were quantified.

$$Recovery rate ( \% ) = [ ( Treatment group swimming distance - Model group swimming distance ) / ( Control group swimming distance - Model group swimming distance ) ] \times 100$$

Control group swimming distance: 1 996.7±263.5 mm.

TABLE 1

| | Swimming | Recovery rate | | ATP | | Lactic Acid | |
|---|---|---|---|---|---|---|---|
| Groups | distance/mm | % | CI | μmol/g | CI | mmol/L | CI |
| Model | 998.3 ± 258.6 | / | / | 814.3 ± 132.8 | / | 0.40 ± 0.01 | / |
| Red bull | 1753.1 ± 413.2*** | 75.6 | / | 935.2 ± 115.3*** | / | 0.31 ± 0.02** | / |
| Red ginseng | 1519.9 ± 365.1** | 52.2 | / | 840.6 ± 121.6* | / | 0.35 ± 0.02* | / |
| Lycium ruthenicum | 1583.5 ± 243.1** | 58.6 | / | 832.7 ± 133.4 | / | 0.34 ± 0.02* | / |
| Composition 1 | 1693.2 ± 269.3** | 69.6 | 0.53 | 893.2 ± 120.5** | 0.74 | 0.31 ± 0.01** | 0.77 |
| Composition 2 | 1790.3 ± 321.5*** | 79.3 | 0.46 | 852.7 ± 145.6* | 0.88 | 0.30 ± 0.02*** | 0.63 |
| Composition 3 | 1655.6 ± 364.3** | 65.6 | 0.71 | 938.4 ± 119.8*** | 0.32 | 0.33 ± 0.02* | 0.86 |

Note:
identical dose for all groups. /: not applicable. Significance vs model:
*p < 0.05,
**p < 0.01,
***p < 0.001.

As shown in Table 1, compared with the control group, the swimming distance of zebrafish in the model group was reduced by 50.0%, confirming that sodium sulfite successfully induced fatigue in zebrafish. Red Bull—an internationally recognized anti-fatigue health drink—was employed as the positive control. The swimming distance of zebrafish in the positive control group increased by 75.6% (p<0.001) compared with the model group, showing a significant recovery. This confirms that Red Bull alleviated sodium sulfite-induced fatigue in zebrafish, verifying the successful establishment of the fatigue model.

Red ginseng and Lycium ruthenicum, when used alone, both significantly increased zebrafish swimming distance (p<0.05) with recovery rates exceeding 50%, demonstrating good anti-fatigue potential. When combined at ratios of 3:4, 2:9, and 8:1, the swimming distance of zebrafish increased by 69.5%, 79.3%, and 65.8% (p<0.01) compared with the model group, with corresponding recovery rates of 69.6%, 79.3%, and 65.6%. The combination index (CI) values were 0.53, 0.46, and 0.71 (all<1), confirming that Red ginseng and Lycium ruthenicum exert synergistic physical fatigue-relieving effects when combined at ratios of 2:9-8:1.

Red ginseng alone increased ATP content (p<0.05), while Lycium ruthenicum showed no significant effect, suggesting Red ginseng alleviates fatigue by boosting ATP levels. When combined at 3:4, 2:9, and 8:1, ATP content increased by 9.6%, 4.7%, and 15.2% (p<0.05) with CI values of 0.74, 0.88, and 0.32 (all<1). This indicates the combinations (2:9-8:1) synergistically elevate ATP content to enhance anti-fatigue efficacy. Both ingredients alone reduced lactic acid content (p<0.05), indicating fatigue relief via lowering bodily lactic acid accumulation. When combined at 3:4, 2:9, and 8:1, lactic acid content decreased by 22.5%, 25.0%, and 17.5% (p<0.05) compared with the model group, with CI values of 0.77, 0.63, and 0.86 (all<1). This confirms the combinations (2:9-8:1) synergistically reduce lactic acid levels to alleviate physical fatigue.

TABLE 2

| | Nrf2 | | atp2b1 | | g6pca | |
|---|---|---|---|---|---|---|
| Groups | Expression | CI | Expression | CI | Expression | CI |
| Model | 0.68 ± 0.29 | / | 0.69 ± 0.41 | / | 0.66 ± 0.17 | / |
| Red bull | 2.74 ± 0.38*** | / | 1.83 ± 0.15** | / | 1.94 ± 0.24** | / |
| Red ginseng | 1.40 ± 0.10* | / | 1.68 ± 1.18* | / | 1.47 ± 0.15* | / |
| Lycium ruthenicum | 1.58 ± 0.22* | / | 1.51 ± 1.00* | / | 1.68 ± 0.05* | / |
| Composition 1 | 3.67 ± 0.37*** | 0.32 | 1.98 ± 0.89** | 0.87 | 1.80 ± 0.25** | 0.71 |
| Composition 2 | 2.57 ± 0.44** | 0.58 | 1.75 ± 0.36* | 0.95 | 1.73 ± 0.54* | 0.88 |
| Composition 3 | 2.18 ± 0.22** | 0.77 | 2.14 ± 0.77** | 0.49 | 1.96 ± 0.66** | 0.64 |

Note:
identical dose for all groups. /: not applicable. Significance vs model:
*p < 0.05,
**p < 0.01,
***p < 0.001.

As shown in Table 2, Red ginseng and Lycium ruthenicum alone both upregulated Nrf2 expression (p<0.05), indicating enhanced antioxidant capacity contributes to their anti-fatigue effects. After combination at 3:4, 2:9, and 8:1, Nrf2 expression increased by 4.39, 2.78, and 2.21 times (p<0.01) with CI values of 0.32, 0.58, and 0.77. This demonstrates the combinations synergistically regulate the antioxidant pathway to relieve fatigue. Both ingredients alone also increased atp2b1 expression (p<0.05). When combined at 3:4, 2:9, and 8:1, atp2b1 expression rose by 1.87, 1.51, and 2.10 times (p<0.05) with CI values of 0.87, 0.95, and 0.49, indicating synergistic regulation of calcium ion metabolism to alleviate fatigue. Additionally, both ingredients alone upregulated g6pca expression (p<0.05). After combination at 3:4, 2:9, and 8:1, g6pca expression increased by 1.72, 1.62, and 1.97 times (p<0.05) with CI values of 0.71, 0.88, and 0.64. This shows the combinations synergistically enhance the energy metabolism pathway to relieve physical fatigue.

In conclusion, Red ginseng and Lycium ruthenicum combined at ratios of 2:9-8:1 synergistically regulate antioxidant capacity, calcium ion metabolism, and energy metabolism. They also synergistically increase ATP content and accelerate lactic acid clearance, further extending zebrafish swimming distance, improving recovery rate, and significantly alleviating physical fatigue—with a recovery effect comparable to that of Red Bull.

### Example 2

The experimental protocol was identical to Example 1. The tested combinations and their ratios are listed in Table 3. Control group swimming distance: 2 020.4±213.6 mm.

TABLE 3

| | Swimming | Recovery rate | | ATP | | Lactic Acid | |
|---|---|---|---|---|---|---|---|
| Groups | distance/mm | % | CI | μmol/g | CI | mmol/L | CI |
| Model | 972.9 ± 362.3 | / | / | 778.6 ± 27.3 | / | 0.38 ± 0.02 | / |
| Red bull | 1745.1 ± 365.4*** | 74.8 | / | 922.1 ± 89.2*** | / | 0.30 ± 0.02** | / |
| Red ginseng | 1558.4 ± 198.7** | 55.9 | / | 828.7 ± 69.5* | / | 0.33 ± 0.01* | / |
| Ganoderma lucidum | 1398.4 ± 253.6* | 39.6 | / | 816.7 ± 78.6* | / | 0.32 ± 0.02* | / |
| Composition 4 | 1492.5 ± 165.9* | 49.5 | 0.79 | 863.4 ± 33.5** | 0.82 | 0.29 ± 0.01** | 0.46 |
| Composition 5 | 1730.1 ± 365.2*** | 73.3 | 0.37 | 840.7 ± 19.3** | 0.91 | 0.27 ± 0.01*** | 0.44 |
| Composition 6 | 1631.3 ± 412.3** | 63.4 | 0.64 | 918.6 ± 12.9*** | 0.28 | 0.31 ± 0.02* | 0.89 |

Note:
identical dose for all groups. /: not applicable. Significance vs model:
*p < 0.05,
**p < 0.01,
***p < 0.001.

As shown in Table 3, Red ginseng and Ganoderma lucidum, when used alone, both increased zebrafish swimming distance (p<0.05), with recovery rates exceeding 30% for both, demonstrating potential for alleviating physical fatigue. When combined at ratios of 3:4, 1:5, and 8:3, the swimming distance increased by 53.4%, 77.8%, and 67.6% (p<0.05) compared with the model group, with corresponding recovery rates of 49.5%, 73.3%, and 63.4%. The respective combination index (CI) values were 0.79, 0.37, and 0.64 (all<1), confirming that Red ginseng and Ganoderma lucidum exert synergistic physical fatigue-relieving effects when combined at ratios of 1:5-8:3.

Red ginseng and Ganoderma lucidum, when used alone, both increased ATP content (p<0.05), indicating that each alleviates fatigue by elevating ATP levels. When combined at ratios of 3:4, 1:5, and 8:3, ATP content increased by 10.9%, 7.9%, and 17.9% (p<0.01), with combination index (CI) values of 0.82, 0.91, and 0.28 (all<1). This confirms that the two ingredients synergistically increase ATP content to relieve fatigue. Both ingredients alone also reduced lactic acid content (p<0.05), thereby alleviating physical fatigue. When combined at 3:4, 1:5, and 8:3, lactic acid content decreased by 23.6%, 28.9%, and 18.4% (p<0.05), with CI values of 0.46, 0.44, and 0.89 (all<1). This demonstrates the combinations synergistically reduce lactic acid accumulation to enhance anti-fatigue efficacy.

TABLE 4

| | Nrf2 | | atp2b1 | | g6pca | |
|---|---|---|---|---|---|---|
| Groups | Expression | CI | Expression | CI | Expression | CI |
| Model | 0.65 ± 0.21 | / | 0.63 ± 0.37 | / | 0.64 ± 0.17 | / |
| Red bull | 2.69 ± 0.33*** | / | 1.91 ± 0.22** | / | 1.89 ± 0.12** | / |
| Red ginseng | 1.28 ± 0.19* | / | 1.55 ± 0.53* | / | 1.35 ± 0.22* | / |
| Ganoderma lucidum | 1.68 ± 0.33* | / | 1.69 ± 1.59** | / | 1.53 ± 0.23* | / |
| Composition 4 | 2.40 ± 0.23*** | 0.61 | 1.89 ± 0.58** | 0.33 | 1.79 ± 0.22** | 0.68 |
| Composition 5 | 1.88 ± 0.33** | 0.88 | 1.77 ± 0.43** | 0.49 | 1.66 ± 0.31* | 0.71 |
| Composition 6 | 2.49 ± 0.62*** | 0.44 | 1.95 ± 0.36** | 0.29 | 1.88 ± 0.33** | 0.45 |

Note:
identical dose for all groups. /: not applicable. Significance vs model:
*p < 0.05,
**p < 0.01,
***p < 0.001.

As shown in Table 4, Red ginseng and Ganoderma lucidum alone both upregulated Nrf2 expression (p<0.05). After combination at 3:4, 1:5, and 8:3, Nrf2 expression increased by 2.69, 1.89, and 2.83 times (p<0.01), with CI values of 0.61, 0.88, and 0.44. This indicates the combinations synergistically regulate the antioxidant pathway to alleviate physical fatigue. Both ingredients alone also increased atp2b1 expression (p<0.05). When combined at the above ratios, atp2b1 expression rose by 2.0, 1.81, and 2.1 times (p<0.01), with CI values of 0.33, 0.49, and 0.29. This confirms the combinations synergistically regulate calcium ion metabolism to relieve fatigue. Additionally, both ingredients alone upregulated g6pca expression (p<0.05). After combination at 3:4, 1:5, and 8:3, g6pca expression increased by 1.79, 1.59, and 1.94 times (p<0.05), with CI values of 0.68, 0.71, and 0.45. This shows the combinations synergistically enhance the energy metabolism pathway to alleviate physical fatigue.

In conclusion, Red ginseng and Ganoderma lucidum combined at ratios of 1:5-8:3 synergistically regulate antioxidant capacity, calcium ion metabolism, and energy metabolism. They also synergistically increase ATP content, accelerate lactic acid clearance, extend zebrafish swimming distance, improve recovery rate, and significantly alleviate physical fatigue—with a recovery effect comparable to that of Red Bull.

### Example 3

The experimental protocol was identical to Example 1. The tested combinations and their ratios are listed in Table 5. Control group swimming distance: 1 594.8±347.6 mm.

TABLE 5

| | Swimming | Recovery rate | | ATP | | Lactic Acid | |
|---|---|---|---|---|---|---|---|
| Groups | distance/mm | % | CI | μmol/g | CI | mmol/L | CI |
| Model | 796.8 ± 198.3 | / | / | 818.6 ± 132.0 | / | 796.8 ± 198.3 | / |
| Red bull | 1329.9 ± 263.5*** | 66.8 | / | 919.8 ± 126.5*** | / | 1329.9 ± 263.5*** | 66.8 |
| Lycium ruthenicum | 1138.0 ± 253.6* | 53.3 | / | 836.6 ± 175.5 | / | 1138.0 ± 253.6* | 53.3 |
| Ganoderma lucidum | 1096.8 ± 189.7* | 37.6 | / | 850.0 ± 166.6* | / | 1096.8 ± 189.7* | 37.6 |
| Composition 7 | 1231.0 ± 198.1** | 55.7 | 0.57 | 883.5 ± 125.6** | 0.44 | 1231.0 ± 198.1** | 55.7 |
| Composition 8 | 1282.9 ± 165.4*** | 61.0 | 0.26 | 928.2 ± 116.5*** | 0.19 | 1282.9 ± 165.4*** | 61.0 |
| Composition 9 | 1262.2 ± 175.1** | 58.4 | 0.69 | 863.5 ± 126.1* | 0.51 | 1262.2 ± 175.1** | 58.4 |

Note:
identical dose for all groups. /: not applicable. Significance vs model:
*p < 0.05,
**p < 0.01,
***p < 0.001.

As shown in Table 5, Ganoderma lucidum and Lycium ruthenicum, when used alone, both increased zebrafish swimming distance (p<0.05), with recovery rates exceeding 30% for both, demonstrating potential for alleviating physical fatigue. When combined at ratios of 3:4, 1:10, and 3:1, the swimming distance increased by 54.49%, 61.0%, and 58.4% (p<0.01) compared with the model group, with corresponding recovery rates of 55.7%, 61.0%, and 58.4%. The respective combination index (CI) values were 0.57, 0.26, and 0.69 (all<1), confirming that Ganoderma lucidum and Lycium ruthenicum exert synergistic physical fatigue-relieving effects when combined at ratios of 1:10-3:1.

Ganoderma lucidum, when used alone, increased ATP content (p<0.05), while Lycium ruthenicum had no significant effect. When combined at ratios of 3:4, 1:10, and 3:1, ATP content increased by 7.9%, 13.3%, and 5.4% (p<0.05), with corresponding combination index (CI) values of 0.44, 0.19, and 0.51 (all<1). This confirms that the combination synergistically increases ATP content to alleviate physical fatigue. Both Ganoderma lucidum and Lycium ruthenicum alone reduced lactic acid content (p<0.05), contributing to physical fatigue relief. When combined at 3:4, 1:10, and 3:1, lactic acid content decreased by 20.5%, 28.2%, and 23.0% (p<0.05), with CI values of 0.56, 0.22, and 0.48 (all<1). This demonstrates the combinations synergistically reduce lactic acid accumulation to enhance anti-fatigue efficacy.

TABLE 6

| | Nrf2 | | atp2b1 | | g6pca | |
|---|---|---|---|---|---|---|
| Groups | Expression | CI | Expression | CI | Expression | CI |
| Model | 0.62 ± 0.28 | / | 0.59 ± 0.23 | / | 0.69 ± 0.22 | / |
| Red bull | 2.83 ± 0.44*** | / | 1.87 ± 0.36** | / | 1.98 ± 0.24** | / |
| Lycium ruthenicum | 1.55 ± 0.55* | / | 1.58 ± 0.74* | / | 1.54 ± 0.12* | / |
| Ganoderma lucidum | 1.63 ± 0.45* | / | 1.71 ± 0.59** | / | 1.49 ± 0.24* | / |
| Composition 7 | 2.59 ± 0.81*** | 0.54 | 1.81 ± 0.71** | 0.76 | 1.96 ± 0.16** | 0.67 |
| Composition 8 | 2.69 ± 0.44*** | 0.39 | 1.91 ± 0.51** | 0.55 | 2.07 ± 0.55** | 0.43 |
| Composition 9 | 2.44 ± 0.79*** | 0.66 | 1.86 ± 0.44** | 0.69 | 1.82 ± 0.66** | 0.77 |

Note:
identical dose for all groups. /: not applicable. Significance vs model:
*p < 0.05,
**p < 0.01,
***p < 0.001.

As shown in Table 6, Ganoderma lucidum and Lycium ruthenicum alone both upregulated Nrf2 expression (p<0.05). After combination at the above ratios, Nrf2 expression increased by 3.17, 3.34, and 2.94 times (p<0.001), with CI values of 0.54, 0.39, and 0.66. This indicates the combinations synergistically regulate the antioxidant pathway to relieve physical fatigue.

Both ingredients alone also increased atp2b1 expression (p<0.05). When combined at 3:4, 1:10, and 3:1, atp2b1 expression rose by 2.06, 2.24, and 2.15 times (p<0.01), with CI values of 0.76, 0.55, and 0.69. This confirms the combinations synergistically regulate calcium ion metabolism to alleviate fatigue.

Additionally, both ingredients alone upregulated g6pca expression (p<0.05). After combination at the tested ratios, g6pca expression increased by 1.84, 2.0, and 1.64 times (p<0.01), with CI values of 0.67, 0.43, and 0.77. This shows the combinations synergistically enhance the energy metabolism pathway to relieve physical fatigue.

In conclusion, Ganoderma lucidum and Lycium ruthenicum combined at ratios of 1:10-3:1 synergistically regulate antioxidant capacity, calcium ion metabolism, and energy metabolism. They also synergistically increase ATP content, accelerate lactic acid clearance, significantly extend zebrafish swimming distance, and improve recovery rate—effectively alleviating physical fatigue with a recovery effect comparable to that of Red Bull.

### Example 4

The experimental protocol was identical to Example 1. The tested formulations and their ratios are listed in Table 7. Control group swimming distance: 1679.1±251.3 mm.

TABLE 7

| | Swimming | Recovery rate | | ATP | | Lactic Acid | |
|---|---|---|---|---|---|---|---|
| Groups | distance/mm | % | CI | μmol/g | CI | mmol/L | CI |
| Model | 839.6 ± 268.3 | / | / | 798.3 ± 17.6 | / | 0.41 ± 0.02 | / |
| Red bull | 1474.4 ± 368.8*** | 76.8 | / | 900.8 ± 26.5*** | / | 0.27 ± 0.03*** | / |
| Red ginseng | 1244.2 ± 263.7* | 48.2 | / | 840.5 ± 33.2* | / | 0.35 ± 0.02* | / |
| Lycium ruthenicum | 1270.2 ± 152.4* | 51.3 | / | 831.5 ± 78.6* | / | 0.35 ± 0.01* | / |
| Ganoderma lucidum | 1162.8 ± 213.5* | 38.5 | / | 845.66 ± 88.5* | / | 0.34 ± 0.02* | / |
| Composition 10 | 1492.6 ± 245.6*** | 77.7 | 0.21 | 936.5 ± 22.4*** | 0.43 | 0.27 ± 0.01*** | 0.38 |
| Composition 11 | 1456.8 ± 453.6** | 73.5 | 0.30 | 895.6 ± 24.3*** | 0.22 | 0.26 ± 0.01*** | 0.33 |
| Composition 12 | 1463.5 ± 369.5** | 74.3 | 0.35 | 906.4 ± 27.6*** | 0.32 | 0.30 ± 0.02** | 0.29 |
| Composition 13 | 1569.2 ± 296.5*** | 86.9 | 0.15 | 984.8 ± 76.9*** | 0.11 | 0.24 ± 0.01*** | 0.13 |

Note:
identical dose for all groups. /: not applicable. Significance vs model:
*p < 0.05,
**p < 0.01,
***p < 0.001.

As shown in Table 7, Red ginseng, Lycium ruthenicum, and Ganoderma lucidum—when used alone—all increased zebrafish swimming distance (p<0.05), with recovery rates of 48.2%, 51.3%, and 38.5% respectively, demonstrating potential for alleviating physical fatigue. When the Red ginseng-Lycium ruthenicum mixture was combined with Ganoderma lucidum at ratios of 3:4, 3:10, and 6:1, the swimming distance increased by 77.7%, 73.5%, and 74.3% (p<0.01) compared with the model group, with corresponding recovery rates of 77.7%, 73.5%, and 74.3%. The respective combination index (CI) values were 0.21, 0.30, and 0.35 (all<1), confirming that Red ginseng, Lycium ruthenicum, and Ganoderma lucidum exert significant synergistic physical fatigue-relieving effects when combined at ratios of 3:10-6:1. All three ingredients alone increased ATP content (p<0.05). When the Red ginseng-Lycium ruthenicum mixture was combined with Ganoderma lucidum at 3:4, 3:10, and 6:1, ATP content increased by 12.1%, 17.3%, and 13.5% (p<0.001), with CI values of 0.43, 0.22, and 0.32 (all<1). This indicates the ternary combination synergistically elevates ATP content to relieve physical fatigue. All three ingredients also reduced lactic acid content (p<0.05), contributing to fatigue relief When combined at the above ratios, lactic acid content decreased by 34.100, 36.5%, and 26.8% (p<0.01) compared with the model group, with CI values of 0.38, 0.33, and 0.29 (all<1). This demonstrates the ternary combination (3:10-6:1) synergistically reduces lactic acid accumulation to enhance anti-fatigue efficacy.

TABLE 8

| | Nrf2 | | atp2b1 | | g6pca | |
|---|---|---|---|---|---|---|
| Groups | Expression | CI | Expression | CI | Expression | CI |
| Model | 0.66 ± 0.31 | / | 0.69 ± 0.12 | / | 0.60 ± 0.15 | / |
| Red bull | 2.78 ± 0.23*** | / | 1.96 ± 0.41*** | / | 1.89 ± 0.32** | / |
| Red ginseng | 1.44 ± 0.22* | / | 1.67 ± 1.25* | / | 1.39 ± 0.31* | / |
| Lycium ruthenicum | 1.34 ± 0.21* | / | 1.51 ± 0.32* | / | 1.59 ± 0.21* | / |
| Ganoderma lucidum | 1.54 ± 0.54* | / | 1.74 ± 1.21** | / | 1.46 ± 0.31* | / |
| Composition 10 | 2.72 ± 0.74*** | 0.36 | 4.26 ± 0.99*** | 0.21 | 2.01 ± 0.49*** | 0.33 |
| Composition 11 | 2.66 ± 0.29*** | 0.46 | 3.65 ± 0.78*** | 0.41 | 1.80 ± 0.63*** | 0.44 |
| Composition 12 | 2.32 ± 0.66*** | 0.55 | 4.06 ± 0.66*** | 0.33 | 1.95 ± 0.72*** | 0.29 |
| Composition 13 | 2.98 ± 0.88*** | 0.21 | 4.59 ± 0.89*** | 0.20 | 2.21 ± 0.69*** | 0.19 |

Note:
identical dose for all groups. /: not applicable. Significance vs model:
*p < 0.05,
**p < 0.01,
***p < 0.001.

As shown in Table 8, Red ginseng, Lycium ruthenicum, and Ganoderma lucidum alone all upregulated Nrf2 expression (p<0.05), indicating they alleviate fatigue by regulating the antioxidant pathway. After combining the Red ginseng-Lycium ruthenicum mixture with Ganoderma lucidum at 3:4, 3:10, and 6:1, Nrf2 expression increased by 3.12, 3.03, and 2.52 times (p<0.001), with CI values of 0.36, 0.46, and 0.55. This confirms the ternary combination synergistically regulates the antioxidant pathway to relieve physical fatigue. All three ingredients alone also increased atp2b1 expression (p<0.05), regulating calcium ion metabolism to alleviate fatigue. When combined at the tested ratios, atp2b1 expression rose by 5.17, 4.29, and 4.88 times (p<0.001), with CI values of 0.21, 0.41, and 0.33. This shows the ternary combination synergistically modulates calcium ion metabolism to relieve physical fatigue. Additionally, all three ingredients alone upregulated g6pca expression (p<0.05), indicating they alleviate fatigue by enhancing the energy metabolism pathway. After combining the Red ginseng-Lycium ruthenicum mixture with Ganoderma lucidum at 3:4, 3:10, and 6:1, g6pca expression increased by 2.35, 2.0, and 2.25 times (p<0.001), with CI values of 0.33, 0.44, and 0.29. This demonstrates the ternary combination significantly synergistically regulates the energy metabolism pathway to alleviate physical fatigue.

In conclusion, the Red ginseng-Lycium ruthenicum mixture combined with Ganoderma lucidum at ratios of 3:10-6:1 synergistically regulates antioxidant capacity, calcium ion metabolism, and energy metabolism. It also synergistically increases ATP content, accelerates lactic acid clearance, significantly extends zebrafish swimming distance, and improves recovery rate-effectively alleviating physical fatigue.

Combined with the experimental results of Examples 1-3, it was found that any binary combination of Red ginseng, Lycium ruthenicum, and Ganoderma lucidum exerts synergistic physical fatigue-relieving effects. Moreover, the ternary combination exhibits a stronger synergistic effect, with a recovery effect slightly superior to that of Red Bull.

### Example 5

Although the Red ginseng-Lycium ruthenicum-Ganoderma lucidum combinations showed clear anti-fatigue activity in zebrafish, anatomical differences between zebrafish and humans require validation in a mammalian model. Therefore, the same combinations were evaluated in mice in accordance with the Methods for Functional Inspection and Evaluation of Health Food (2023 Edition, State Administration for Market Regulation of the People's Republic of China) to generate data directly relevant to product development. The synergistic doses identified in zebrafish were converted to mouse equivalents using the interspecies scaling factor described in CN113496071A.

Male C57BL/6J mice (20±2 g) were gavaged daily for 30 days (test groups are listed in Table 9). Thirty minutes after the final gavage, a weight-loaded (5% of body weight) forced-swim test was conducted. Exhaustion time was recorded as the interval until the mouse remained submerged for 5 seconds. Serum and tissues were then collected to assay lactic acid, serum urea nitrogen (BUN), muscle glycogen, and hepatic glycogen levels.

TABLE 9

| | | | Urea | Muscle | Hepatic |
|---|---|---|---|---|---|
| | Swimming | Lactic Acid | nitrogen | glycogen | glycogen |
| Groups | time/min | (mmol/L) | (mmol/L) | (mg/g) | (mg/g) |
| Model | 5.36 ± 1.36 | 7.35 ± 1.58 | 10.75 ± 0.94 | 0.80 ± 0.16 | 4.37 ± 0.81 |
| Red bull | 9.86 ± 3.09** | 5.22 ± 1.28*** | 8.82 ± 0.98** | 1.12 ± 0.23** | 5.81 ± 1.40** |
| Composition | 9.87 ± 2.36** | 4.69 ± 0.99*** | 9.01 ± 1.04* | 1.14 ± 0.12** | 5.78 ± 0.87** |
| 10 | | | | | |
| Composition | 9.58 ± 2.06* | 4.77 ± 1.37*** | 9.26 ± 1.46* | 0.97 ± 0.25* | 4.97 ± 1.11* |
| 11 | | | | | |
| Composition | 8.94 ± 2.45* | 5.26 ± 0.99** | 9.12 ± 0.77* | 0.95 ± 0.17* | 5.15 ± 0.97* |
| 12 | | | | | |
| Composition | 10.87 ± 2.12** | 4.22 ± 1.28*** | 8.92 ± 0.67** | 1.22 ± 0.34** | 5.96 ± 2.10** |
| 13 | | | | | |

Note:
equal dose for all groups. Significance vs model:
*p < 0.05,
**p < 0.01,
***p < 0.001.

As shown in Table 9, Red Bull—an internationally recognized anti-fatigue health drink—was used as the positive control. The exhaustion swimming time of mice in the positive control group was extended by 83.9% (p<0.01), confirming that Red Bull significantly alleviates physical fatigue in mice. When the Red ginseng-Lycium ruthenicum mixture was combined with Ganoderma lucidum powder at ratios of 3:4, 3:10, and 6:1, the exhaustion swimming time of mice was extended by 84.14%, 78.73%, and 66.79% (p<0.05), respectively. This demonstrates that the formula exerts physical fatigue-relieving efficacy in mice when the Red ginseng-Lycium ruthenicum: Ganoderma lucidum ratio ranges from 3:10-6:1. At the aforementioned ratios, the lactic acid content decreased by 36.1%, 35.1%, and 28.4% (p<0.001), while the serum urea nitrogen (BUN) content decreased by 16.1%, 13.8%, and 15.1% (p<0.05). This confirms that the formula (ratio 3:10-6:1) significantly alleviates physical fatigue by reducing bodily accumulation of lactic acid and serum urea nitrogen. Additionally, at the ratios of 3:4, 3:10, and 6:1, the muscle glycogen content increased by 42.5%, 21.2%, and 18.7% (p<0.05), and the hepatic glycogen content increased by 32.2%, 13.7%, and 17.8% (p<0.05), respectively. This shows that the formula (ratio 3:10-6:1) alleviates physical fatigue by increasing the body's glycogen reserves.

In conclusion, the Red ginseng-Lycium ruthenicum mixture combined with Ganoderma lucidum extract powder at ratios of 3:10-6:1 significantly extends the exhaustion swimming time of mice by reducing metabolite accumulation and increasing glycogen levels. This indicates the formula effectively alleviates physical fatigue in mice, with a recovery effect slightly superior to that of Red Bull. To date, the composition of the present invention has been verified to have significant physical fatigue-alleviating efficacy, in compliance with the standard methods specified in the Methods for Functional Inspection and Evaluation of Health Food (2023 Edition, State Administration for Market Regulation of the People's Republic of China).

### COMPARATIVE EXAMPLES

### Comparative Compositions

Comparative Composition 1: Lycium barbarum fruit powder

Comparative Composition 2: Hippophae rhamnoides (sea-buckthorn) fruit powder

Comparative Composition 3: Polygonatum (Huangjing) extract powder

Comparative Composition 4: 1 part Lycium barbarum extract powder+2 parts Hippophae rhamnoides extract powder

Comparative Composition 5: 1 part Lycium barbarum extract powder+3 parts Polygonatum extract powder

Comparative Composition 6: 2 parts Hippophae rhamnoides extract powder+3 parts Polygonatum extract powder

Comparative Composition 7: 1 part Lycium barbarum extract powder, 2 parts Hippophae rhamnoides extract powder, and 3 parts Polygonatum extract powder

The above comparative formulations were tested in zebrafish using the same protocol as the working examples. Results are summarized in Table 10.

TABLE 10

| | Swimming | Recovery | |
|---|---|---|---|
| Groups | distance/mm | rate/% | CI |
| Blank | 2135.6 ± 364.8 | / | / |
| Model | 1086.4 ± 189.5 | / | / |
| Comparative Composition 1 | 1362.1 ± 223.6 | 26.3 | / |
| Comparative Composition 2 | 1394.7 ± 253.4 | 29.3 | / |
| Comparative Composition 3 | 1333.6 ± 403.8 | 23.6 | / |
| Comparative Composition 4 | 1408 ± 269.5 | 30.7 | 1.78 |
| Comparative Composition 5 | 1423 ± 333.8 | 32.1 | 1.69 |
| Comparative Composition 6 | 1505 ± 369.5 | 39.9 | 1.43 |
| Comparative Composition 7 | 1522 ± 298.7 | 41.5 | 1.55 |

As shown in Table 10, each individual material exhibited modest anti-fatigue potential, but their binary or ternary combinations yielded virtually no additional improvement in recovery rate. The calculated CI values for these combinations were 1.78, 1.69, 1.43, and 1.55 (all>1), indicating antagonistic or merely additive effects. These data confirm that simply combining individual anti-fatigue food ingredients does not guarantee synergistic effects; rational, evidence-driven formulation is therefore essential to achieve genuine efficacy enhancement.

The foregoing examples are merely preferred embodiments of the present invention and are not intended to restrict its scope. Any modifications, equivalent substitutions, or improvements made within the spirit and principles of the present invention shall be included within the scope of protection claimed herein.

1. A use of a traditional Chinese medicinal composition in the preparation of a product for relieving physical fatigue, characterized in that the components consist of Red ginseng and Lycium ruthenicum, and in parts by weight; 2 parts of Red ginseng and 9 parts of Lycium ruthenicum; or
consist of Red ginseng and Ganoderma lucidum, and in parts by weight: 1 part of Red ginseng and 5 parts of Ganoderma lucidum, or
consist of Lycium ruthenicum and Ganoderma lucidum, and in parts by weight: 1-9 parts of Lycium ruthenicum and 3-10 parts of Ganoderma lucidum, or
consist of Red ginseng, Lycium ruthenicum and Ganoderma lucidum, and in parts by weight: 2-8 parts of Red ginseng, 1-9 parts of Lycium ruthenicum and 3-10 parts of Ganoderma lucidum.

2-5. (canceled)

6. The use according to claim 1, characterized in that it consists of the following components in parts by weight: 2 parts of Red ginseng, 3 parts of Lycium ruthenicum and 8 parts of Ganoderma lucidum.

7-9. (canceled)

10. The use according to claim 2, characterized in that the product is in the form of tablets, oral liquids, capsules, granules, or pills.
